(19) United States
(12) Patent Application Publication
(10) Pub. No.: US 2009/0304823 A1
(43) Pub. Date: Dec. 10, 2009

(54) NUTRITIONAL COMPOSITIONS FOR PROMOTION OF BONE GROWTH AND MAINTENANCE OF BONE HEALTH AND METHODS REGARDING SAME

(75) Inventors: Elizabeth Offord Cavin, Montreux (CH); Gary Williamson, Mezieres (CH); Didier Courtois, St-Avertin (FR); Bernard Lemaure, Tours (FR); Andre Touche, Monts (FR); Grace Ing Soon, Singapore (SG); Laurent Ameye, Lausanne (CH)

Correspondence Address: K&L Gates LLP, P.O. Box 1135, CHICAGO, IL, 60690, US

(73) Assignee: NESTEC S.A., Vevey (CH)

(21) Appl. No.: 12/295,918

(22) PCT Filed: Apr. 3, 2007
(86) PCT No.: PCT/EP2007/053214

§ 371 Date: Oct. 3, 2008

Related U.S. Application Data
(63) Continuation-in-part of application No. 60/744,176, filed on Apr. 3, 2006.

(51) Int. Cl. A61K 36/53; A61K 36/00; A61K 31/352; A61P 19/00

(52) U.S. Cl. 424/725; 514/456; 514/453

(57) ABSTRACT

Compositions and methods for maintenance of bone health or prevention, alleviation and/or treatment of bone disorders are presented. The present invention also provides the manufacture of a nutritional product, a supplement or a medicament for promoting bone growth or for the maintenance of bone health and methods regarding same. In an embodiment, the present invention provides a composition comprising an active ingredient having an effective amount of a plant or plant extract containing at least one phytochemical having the ability to induce bone morphogenic protein expression.

### BACKGROUND

The present invention generally relates to nutritional compositions that provide health benefits. More specifically, the present invention relates to beneficial compositions that can be used, for example, to improve bone density and formation and methods regarding same.

Bone mass evolves throughout life and is regulated by genetic, mechanical and hormonal mechanisms. Bone mineral acquisition occurs during childhood and peak bone mass is achieved around 20 years of age. During this period, bone formation exceeds bone resorption. Later in life, and particularly around the time of the menopause, or in the elderly population, bone mass and quality are impaired due to a higher bone turnover with excessive bone resorption leading to a gradual loss of bone mass, microarchitecture, structure and strength. To maintain bone, it is important to restore the balance between bone formation and bone resorption. This bone remodelling process is regulated at the bone cell level involving a tight interaction between bone forming cells (osteoblasts) and bone resorbing cells (osteoclasts).

Phytonutrients, especially flavonoids, can positively influence the bone remodelling process. The most reported data is for the soy isoflavones which, in some studies, have been shown to prevent bone loss and improve bone mineral density (BMD) in postmenopausal women at doses of 50-90 mg/day. However, not all studies with isoflavones are positive and controversy still exists over their efficacy. Further, some epidemiological evidence exists for benefits of tea as a previous study showed that tea drinkers had a higher mean BMD than non tea-drinkers in an elderly population, however no intervention studies have been carried out to substantiate this finding.

Currently, there is a strong interest in identifying agents which can stimulate bone formation. The delivery of recombinant BMP-2 has been shown to induce bone or cartilage formation. For example, bone morphogenic protein 2 (BMP-2) is a member of the TGFβ family and is a key regulator of bone growth during embryonic development, and further bone growth and repair. Statins (effective drugs for cholesterol-lowering through inhibition of the enzyme HMG-CoA reductase) improve bone formation, partly mediated by induction of BMP-2 (G. Mundy, et al., Science 286: 1946-1949 (1999); C. J. Edwards, et al., Lancet, 355: 2218-2219 (2000)). Statins were also able to reduce hip fracture risk in menopausal women (P. S. Wang, et al., JAMA 283 :3211-3216 (2000)).

### SUMMARY

The present invention generally relates to nutritional compositions for maintenance of bone health or prevention, alleviation and/or treatment of bone disorders. The present invention also provides the manufacture of a nutritional product, a supplement or a medicament for promoting bone growth or for the maintenance of bone health and methods regarding same. In particular, the present invention provides the manufacture of a nutritional product, a supplement or a medicament for promoting bone formation which is important for bone growth as well as for the maintenance of bone health through balanced bone remodeling and methods regarding same.

In an embodiment, the present invention provides a composition comprising an active ingredient having an effective amount of a plant or plant extract containing at least one phytochemical having the ability to induce bone morphogenic protein expression.

In an embodiment, the plant or plant extract further inhibits bone resorption.

In an embodiment, the plant is rosemary or caraway.

In an embodiment, the phytochemical is selected from the group consisting of eupafolin, carnosol, scutellarein, genkwanin, kaempferol, acacetin, rosmarinic acid, rosmanol, cirsimaritin, luteolin, 7-epirosmanol, and the compound C-0063-W-06 of FIG. 7A and combinations thereof.

In an embodiment, the composition can be in a form selected from the group consisting of a nutritionally balanced food, pet food, a dietary supplement, a treat, a pharmaceutical composition and combinations thereof.

In an embodiment, the composition can be designed to assist bone regeneration during fracture healing, increase bone formation and bone mineral density during growth and optimize peak bone mass or to decrease bone loss, in particular bone loss associated with age in humans or pets.

In an embodiment, the composition can be designed to build cartilage in humans or pets.

In an embodiment, the composition can be designed to prevent osteoarthritis in humans or pets.

In another embodiment, the present invention provides a composition comprising an active ingredient having an effective amount of a rosemary plant or rosemary plant extract containing at least one phytochemical having the ability to induce bone morphogenic protein expression. For example, the phytochemical can be selected from the group consisting of eupafolin, carnosol, scutellarein, genkwanin, kaempferol, acacetin and combinations thereof.

In an alternative embodiment, the present invention provides a method for manufacturing a food composition for the prevention, the alleviation and/or the treatment of bone disorders or maintenance of bone health in humans or pets, the method comprising providing a food composition; and adding to the food composition an active ingredient having a plant or a plant extract containing at least one phytochemical having the ability to stimulate bone morphogenic protein and/or inhibit bone resorption to prepare the composition. For example, the composition can include components chosen from the group consisting of chicory, tea, cocoa, bioactives, antioxidants, fatty acids, prebiotic fibers, glucosamine, chondroitin sulphate and combinations thereof.

In another embodiment, the present invention provides a method for the treatment, alleviation or prevention of bone disorder or maintenance of bone health, the method comprising administering a therapeutically-effective amount of a composition comprising an active ingredient having an effective amount of at least one plant or plant extract containing at least one phytochemical having the ability to induce bone morphogenic protein expression to an individual in need of same.

In an alternative embodiment, the present invention provides a method of increasing bone formation, bone mineral density during growth and optimize peak bone mass in humans or pets, the method comprising feeding an individual, a composition comprising an active ingredient having an effective amount of at least one plant or plant extract containing at least one phytochemical having the ability to induce bone morphogenic protein expression.

In another embodiment, the present invention provides a method for the treatment, alleviation and/or prophylaxis of osteoarthritis in pets and humans, the method comprising feeding an individual having or at risk of osteoarthritis, a composition comprising an active ingredient having an effective amount of at least one plant or plant extract containing at least one phytochemical having the ability to induce bone morphogenic protein expression in the individual.

In still another embodiment, the present invention provides a method of treating or preventing osteoporosis, the method comprising administering to an individual having or at risk of osteoporosis a therapeutically effective amount of a composition comprising an active ingredient having an effective amount of at least one plant or plant extract containing at least one phytochemical having the ability to induce bone morphogenic protein expression in the individual.

In yet an alternative embodiment, the present invention provides a method of stimulating bone regeneration during fracture healing, the method comprising feeding an individual having a fracture, a therapeutically-effective amount of a composition comprising an active ingredient having an effective amount of at least one plant or plant extract containing at least one phytochemical having the ability to induce bone morphogenic protein expression in the individual.

In a further embodiment, the present invention provides a method of decreasing bone loss, the method comprising feeding an individual exhibiting a bone loss, a composition comprising an active ingredient having an effective amount of at least one plant or plant extract containing at least one phytochemical having the ability to induce bone morphogenic protein expression in the individual.

Additional features and advantages are described herein, and will be apparent from, the following Detailed Description and the figures.

### BRIEF DESCRIPTION OF THE FIGURES

FIG. 1 illustrates an extraction protocol.

FIG. 2 illustrates a summary of the extraction procedure and first fractionation.

FIG. 3A illustrates BMP-2 results for rosemary and caraway extracts.

FIG. 3B illustrates alkaline phosphatase results for rosemary and caraway extracts.

FIG. 3C illustrates bone formation in organ culture for rosemary and caraway extracts.

FIG. 4 illustrates an extraction procedure from NRC initial MeOH/water extract, and BMP-2 activity of the extracts (from 80 g dried leaves (hexane=6 g)).

FIG. 5 illustrates bone formation in vivo with rosemary extract.

FIG. 6A illustrates phenolics found positive in BMP-2 assay.

FIG. 6B illustrates an alkaline phosphatase assay of BMP-2 positive phenolics.

FIG. 6C illustrates an organ culture bone formation: examples with eupafolin and carnosol.

FIGS. 7A-B illustrate compounds isolated from rosemary extract 2188.

FIG. 8A gives details of the effects of rosemary extract on the activity of human osteoclasts.

FIGS. 9A A, B and C give details on the effects of rosemary extract and carnosol on articular cartilage metabolism

FIG. 10 shows Osteopontin (OPN) mRNA induction in human osteoblast cells (hPOBtert) by rosemary extract or carnosol.

FIG. 11 shows that Carnosol induces the expression of the phase II enzyme NQO1, a typically Nrf-1 regulated gene/protein.

### DETAILED DESCRIPTION

The present invention relates to beneficial compositions that can be used, for example, to improve bone density and formation and methods regarding same. For example, in an embodiment, the present invention is directed to plants and plant extracts that stimulate bone formation and improve bone maintenance.

In plants, a number of isoprenoids (monoterpenes, sesquiterpenes, etc.) are modulators of both HMG-CoA reductase and protein prenylation, mechanisms probably linked to either the bone resorption inhibition or bone formation enhancement. Therefore, certain plant compounds can be potential inhibitors of bone resorption and/or enhancers of bone formation.

In embodiments of the present invention, extracts were prepared from edible and/or medicinal plant species, which were proposed based on potential benefits for relief of menopausal symptomes or their capacity of affecting the cholesterol synthesis pathway and therefore having a potential to stimulate BMP-2 and bone formation. As discussed in more detail later, extracts were generally prepared by a 4-step process (a) hexane, (b) methanol-water, (c) methanol-water extracts hydrolyzed with glycosidases and re-extracted with ethylacetate, and (d) removal of large polyphenols with a PVPP column. The methanol-water and ethylacetate extracts were used for in vitro screening. Extracts were hydrolyzed by α- and β-glycosidases instead of acid to ensure release of flavonoid aglycones (biologically active form) from their glycosides.

The following bioassays were used for bone formation analysis

BMP-2 gene reporter assay (high throughput screen)

Alkaline phosphatase in osteoblast cells

Calvaria organ culture in vitro, bone formation

Injection into calvaria in vivo, bone formation.

For example, extracts were screened for bone formation by a high throughput gene reporter assay for BMP-2 followed by alkaline phosphatase assay and an organ culture model and finally injection into mice calvaria in vivo. Subfractions of positive extracts and/or pure compounds were further tested for activity. Analysis of the chemical composition of one active extract was performed in order to determine the active compounds.

It was surprisingly found that compounds extracted from rosemary plants could be used as active compounds for bone development, growth and/or maintenance. For example, at least 3 phenolics (e.g. eupafolin, carnosol, scutellarein) may contribute to the anabolic potential of the rosemary extract. Additional phenolics of rosemary include genkwanin, kaempferol, acacetin. The rosemary plant extracts and pure compounds were tested in an osteoblast/osteoclast co-culture system. The rosemary extract as well as the same 3 phenolics were shown to have an activity for regulation of the key cytokines controlling bone remodelling, i.e. OPG/RANKL. Furthermore, it was found that rosemary extract and carnosol stimulate osteopontin (OPN) expression in human osteoblast cells, possibly via AP-1/Nrf-2 signalling pathways.

Three constituents of the active rosemary extract are new, and have never been described in the literature. Moreover, as several compounds belong to a same chemical class, a cross-linked biological evaluation of these constituents might lead to an interesting structure—activity correlation (e.g. carnosol/rosmanol/isorosmanol) which could provide further strategies for the rosemary extracts but also for other plant species containing the known most active constituents.

In an embodiment of the present invention, the MeOH/water extracts of the rosemary plant (25% of the initial leaf dry matter) obtained after a defatting step with hexane contain the molecules responsible for the activity and could be used in a food product. The in vitro activity is only slightly observed in the initial MeOH/water extract due to the dilution of active compounds by other compounds and also for a part due to the presence of bound forms. High activity is observed on BMP-2 assay after purification/concentration through ethyl acetate extraction and/or hydrolysis with glucosidase then ethyl acetate extraction.

The plants or plant extracts according to embodiments of the present invention may be used in the preparation of a food composition. The composition may be in the form of a nutritionally balanced food or pet food, a dietary supplement, a treat or a pharmaceutical composition.

The plant or plant extract may be used alone or in association with other plants such as chicory, tea, cocoa, or with other bioactive molecule such as antioxidants, fatty acids, prebiotic fibers, glucosamine, chondroitin sulphate, for example.

In one embodiment of the present invention, a food composition or nutritional formula for human consumption is prepared. This composition may be a nutritional complete formula, a dairy product, a chilled or shelf stable beverage, soup, a dietary supplement, a meal replacement, and a nutritional bar or a confectionery.

Apart from the rosemary plant or rosemary plant extract according to the invention, the nutritional formula may comprise a source of protein. Dietary proteins are preferably used as a source of protein. The dietary proteins may be any suitable dietary protein; for example animal proteins (such as milk proteins, meat proteins and egg proteins); vegetable proteins (such as soy protein, wheat protein, rice protein, and pea protein); mixtures of free amino acids; or combinations thereof. Milk proteins such as casein, whey proteins and soy proteins are particularly preferred. The composition may also contain a source of carbohydrates and a source of fat.

If the nutritional formula includes a fat source, the fat source preferably provides about 5% to about 55% of the energy of the nutritional formula; for example about 20% to about 50% of the energy. The lipids making up the fat source may be any suitable fat or fat mixtures. Vegetable fats are particularly suitable; for example soy oil, palm oil, coconut oil, safflower oil, sunflower oil, corn oil, canola oil, lecithins, and the like. Animal fats such as milk fats may also be added if desired.

A source of carbohydrate may be added to the nutritional formula. It preferably provides about 40% to about 80% of the energy of the nutritional composition. Any suitable carbohydrates may be used, for example sucrose, lactose, glucose, fructose, corn syrup solids, and maltodextrins, and mixtures thereof. Dietary fiber may also be added if desired. If used, it preferably comprises up to about 5% of the energy of the nutritional formula. The dietary fiber may be from any suitable origin, including for example soy, pea, oat, pectin, guar gum, gum arabic, and fructooligosaccharides. Suitable vitamins and minerals may be included in the nutritional formula in an amount to meet the appropriate guidelines.

One or more food grade emulsifiers may be incorporated into the nutritional formula if desired; for example diacetyl tartaric acid esters of mono- and di-glycerides, lecithin and mono- and di-glycerides. Similarly suitable salts and stabilizers may be included. Vitamins and minerals may also be combined with the plant extract.

The nutritional composition is preferably enterally administrable; for example in the form of a powder, tablet, capsule, a liquid concentrate, solid product or a ready-to-drink beverage. If it is desired to produce a powdered nutritional formula, the homogenized mixture is transferred to a suitable drying apparatus such as a spray drier or freeze drier and converted to powder.

In another embodiment, a nutritional composition comprises a milk-based cereal together with a prebiotic formulation. Preferably the milk-based cereal is an infant cereal which acts as a carrier for the prebiotic formulation.

In another embodiment, a usual food product may be enriched with at least one plant or plant extract according to the present invention. For example, a fermented milk, a yoghurt, a fresh cheese, a renneted milk, article of confectionery, for example a sweet or sweetened beverage, a confectionery bar, breakfast cereal flakes or bars, drinks, milk powders, soy-based products, non-milk fermented products or nutritional supplements for clinical nutrition.

The amount of the plant or plant extract in the composition may vary according to the plant source and its utilization. In a preferred embodiment, an efficient daily dose amount is of at least about 1 mg, and more preferably from 1 mg to 200 mg of the active molecule per day.

In an alternative embodiment, a pharmaceutical composition containing at least a rosemary extract or phytochemical as described above, in an amount sufficient to achieve the desired effect in an individual can be prepared. This composition may be a tablet, a liquid, capsules, soft capsules, pastes or pastilles, gums, or drinkable solutions or emulsions a dried oral supplement, a wet oral supplement. The pharmaceutical composition can further contain carriers and excipients that are suitable for delivering the respective active molecule of different nature to the target tissue. The kind of the carrier/excipient and the amount thereof will depend on the nature of the substance and the mode of drug delivery and/or administration contemplated. It will be appreciated that the skilled person will, based on his own knowledge select the appropriate components and galenic form.

The plant or plant extract according to the invention may be used in the preparation of a pet food composition. The said composition may be administered to the pet as a supplement to its normal diet or as a component of a nutritionally complete pet food, and more preferably in an hypocaloric pet food. It may also be a pharmaceutical composition.

The plant or plant extract may be used alone or in association with other plants such as chicory, tea, cocoa, or with other bioactive molecule such as antioxidants, fatty acids, prebiotic fibers, glucosamine, chondroitin sulphate for example.

Preferably, the pet food composition contains about 0.01 to 0.5 g of dry plants per gram of dry pet food for a 15 kg dog; and 0.001 to 0.1 g of dry plants per gram of wet pet food for a 15 kg dog. The nutritionally complete pet food composition according to the invention may be in powdered, dried form, a treat or a wet, chilled or shelf stable pet food product. It may be chilled or provided as a shelf stable product. These pet foods may be produced by ways known in the art.

The pet food may optionally also contain a prebiotic, a probiotic microorganism or another active agent, for example a long chain fatty acid. The amount of prebiotic in the pet food is preferably less than 10% by weight. For example, the prebiotic may comprise about 0.1% to about 5% by weight of the pet food. For pet foods which use chicory as the source of the prebiotic, the chicory may be included to comprise about 0.5% to about 10% by weight of the feed mixture; more preferably about 1% to about 5% by weight.

If a probiotic micro-organism is used, the pet food preferably contains about 10⁴ to about 10¹⁰ cells of the probiotic micro-organism per gram of the pet food; more preferably about 10⁶ to about 10⁶ cells of the probiotic micro-organism per gram. The pet food may contain about 0.5% to about 20% by weight of the mixture of the probiotic micro-organism; preferably about 1% to about 6% by weight; for example about 3% to about 6% by weight.

If necessary, the pet food cam be supplemented with minerals and vitamins so that they are nutritionally complete. Further, various other ingredients, for example, sugar, salt, spices, seasonings, flavoring agents, and the like may also be incorporated into the pet food as desired.

In another embodiment, dietary adjuncts may be prepared so as to improve pet food quality. As dietary adjuncts, they may be encapsulated or may be provided in powder form and packaged in conjunction with or separately from a main meal, be it wet or dry. By way of example, a powder containing extracts according to the invention, may be packed in sachets in a powder form or in a gel or lipid or other suitable carrier. These separately packaged units may be provided together with a main meal or in multi-unit packs for use with a main meal or treat, according to user instructions.

The amount of pet food to be consumed by the pet to obtain a beneficial effect will depend on the size of the pet, the type of pet, and age of the pet. However, an amount of the pet food to provide a daily amount of about 0.5 to 5 g of dry plants per kg of body weight, would usually be adequate for dogs and cats.

Administering to a human or animal, the food or pet food composition as described above, can result in an improved bone regeneration during fracture healing. It can help to stimulate bone formation and bone mineral density during growth and optimize peak bone mass. In particular, it may provide an optimal bone growth during childhood. This food composition can help to prevent bone loss, in particular bone loss associated with age in mammals or bone loss associated with long term hospitalization. It can reduce risk of osteoporosis and improve recovery after fracture. Furthermore, it can help to build cartilage in mammals and prevent osteoarthritis in pets and humans, which results in a better activity or mobility of the individual (e.g. pets and/or humans).

### EXAMPLES

By way of example and not limitation, the following examples are illustrative of various embodiments of the present invention and further illustrate experimental testing conducted in accordance with embodiments of the present invention.

### Example 1

Selected Plants and Pure Compounds

Plants were selected based on knowledge of their phenolics:

(i) 25 plants in addition to soy were selected from a Database of Plant Containing Estrogenic Substances (Table 1A)

(ii) 9 plants+soy containing substances affecting the cholesterol synthesis pathway and thereby having the potential to indirectly stimulate BMP-2 and bone formation (Table 1B)

(iii) Pure compounds: estradiol-17β, genistein, daidzein, 20 flavonoids, chicory compounds (sesquiterpene lactones)

TABLE 1A
List of plant species selected from estrogenic plant database

| NAME | GENERA | SPECIES | ORGAN |
|---|---|---|---|
| Soy | Glycine | max | seeds |
| sweet flag | Acorus | calamus | rhizomes |
| saskatoonberry | Amelanchier | ovalis | fruits |
| mugwort wormwood | Artemisia | vulgaris | aerial part |
| | Cnidium | monnieri | seeds |
| nut grass | Cyperus | rotundus | rhizomes |
| clammy hop seed bush | Dodonaeae | viscosa red | leaves/fruits |
| clammy hop seed bush | Dodonaeae | viscosa yellow | leaves/fruits |
| Yin Yang Huo | Epimedium | brevicornum | aerial parts |
| Saint Catherine's Lace | Eriogonum | giganteum | stem/leaf/root |
| Iris | Iris | pallida | rhizomes |
| Iris | Iris | pallida | cell culture |
| Iris | Iris | pseudacorus | leaves/seeds |
| Juniper | Juniperus | communis | fruit/leaf/wood |
| | Lecaniodiscus | cupianoides | leaves/seeds |
| spice bush | Lindera | benzoin | leaves/fruits |
| basil sp. | Ocimum | gratissimum | leaves/seeds |
| Sourwood | Oxydendrum | arboreum | leaves |
| great plantain | Plantago | major | leaves |
| Peach | Prunus | persica | seeds |
| smooth sumac | Rhus | glabra | leaves/fruits |
| Skullcap | Scutellaria | costaricana | plant |
| Skullcap | Scutellaria | baicalinensis | plant |
| common tansy | Tanacetum | vulgare | leaves |
| common dandelion | Taraxacum | officinalis | roots |
| Ironweed | Vernonia | cinerea | leaves |

TABLE 1B
List of plant species selected for potential effect on
cholesterol and BMP-2

| NAME | GENERA | SPECIES | ORGAN |
|---|---|---|---|
| Soy | Glycine | max | seeds |
| Chicory | Cichorium | intybus | roots |
| Rosemary | Rosmarinus | officinalis | leaves |
| Thyme | Thyme | leaves | |
| Grape | Vitis | vinifera | fruits |
| Caraway | Carum | carvi | seeds |
| Rocou, annatto | Bixa | orellana | seeds |
| Aneth | Anethum | graveolens | leaves |
| Spearmint | Mentha | spicata | leaves |
| Cumin | Cuminum | cyminum | seeds |

TABLE 1C
List of pure compounds

| Plant Source | Pure Compound |
|---|---|
| | Estradiol |
| Soy | Genistein |
| Soy | Daidzein |
| Soy | Equol |
| rosemary | Carnosol |
| Lignans | Enterolactone |
| Lignans | Enterodiol |
| rosemary | carnosic acid |
| rosemary | Eupafolin |
| rosemary | Genkwanin |
| rosemary | Luteolin |
| rosemary | Scutellarein |
| rosemary | Rosmarinic acid |
| | Acacetin |
| | Kaempferol |
| Onion, apple | Quercetin |
| Tea | Catechin |
| Tea | Epicatechin |
| | Apigenin |
| Orange | Hesperetin |
| grapefruit | Naringenin |
| Grape | Resveratrol |
| | Phloretin |
| | Diosmetin |
| | Diosmin |
| | Cirsimaritin |
| | Hispidulin |
| Chicory | Lactucin |
| | Lactucopicrin |
| | 3-deoxylactucin |
| | Geraniol |
| | Carvone |

Plant Extraction Procedure

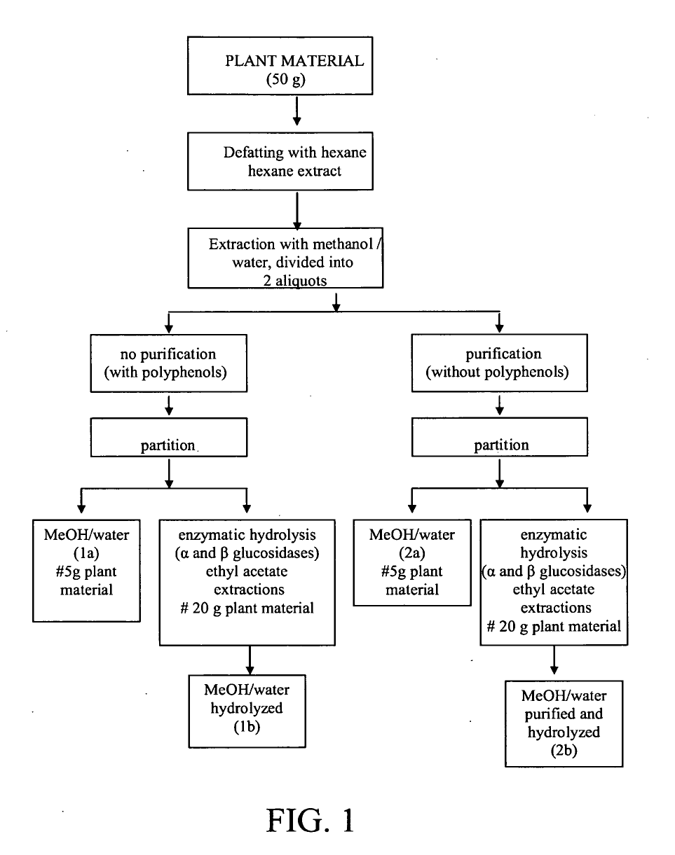

Referring to FIG. 1, the extraction procedure generally included the following steps:

Hexane, defatting (extracts not screened)

MeOH/H₂O (1a)

MeOH/H₂O, hydrolyzed with α, β-glycosidases, extracted with ethylacetate (1b)

1a purified on PVPP column to remove high mol wt polyphenols (2a)

1b purified on PVPP column to remove high mol wt polyphenols (2b)

Extracts 2a and 2b gave similar results to extracts 1a and 1b and therefore the polyphenol purification step was subsequently discontinued. The extraction procedure included glycosidase treatment (instead of acid hydrolysis) to ensure conversion of flavonoid glycosides to aglycones.

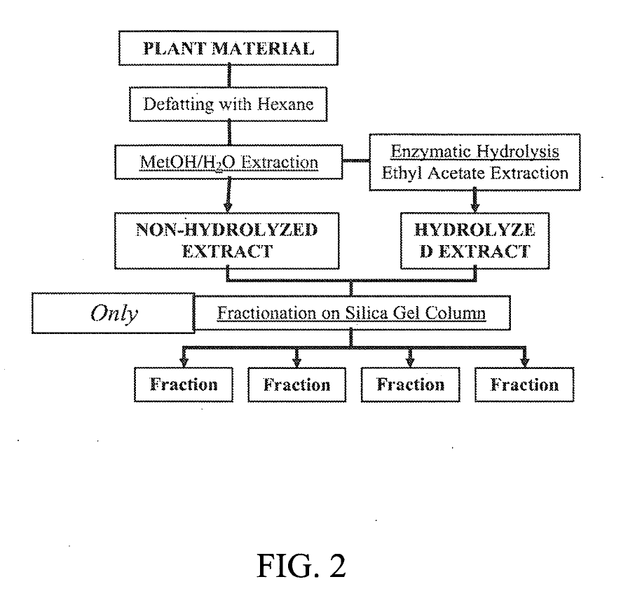

Subfractionation—Four subfractions were prepared by fractionation on silica gel cartridge with elution by solvents of varying polarity: ethyl acetate then ethyl acetate/methanol (95/5) followed by ethyl acetate/methanol (50/50) and finally methanol (FIG. 2).

Screening Steps and Bioassays

The screening for bone formation was carried out in several stages:

(i) BMP-2 high throughput gene reporter assay of non-hydrolyzed MeOH/H₂O extracts (1a) and corresponding glycosidase hydrolyzed extract in ethyl acetate (1b). Extracts were tested twice at concentrations of 1 to 100 μg/ml, diluted in culture medium from plant extract stocks prepared as 50 mg/ml in DMSO.

(ii) Extracts positive in the BMP-2 screen were prepared de novo and screened again with dose-response to confirm as “hits”.

(iii) BMP-2 testing of subfractionation of positives/hits and candidate pure compounds

(iv) Extracts shown positive in the BMP-2 assay were further tested for osteoblast differentiation using the alkaline phosphatase assay and in an organotypic model of bone formation using culture of calvarial bones and histomorphometry for demonstration of bone formation as described by Traianedes et al, (1998).

(v) Final injection in vivo of “hit” extract into mouse calvaria bones and monitoring of bone area and thickness. Extracts were assessed in a 4-day in vitro neonatal murine calvarial assay. Bones were incubated with the extracts for the entire 4 days.

(vi) Resorption activity monitored by measuring the amount of type I collagen released in the media as osteoclasts digest bone.

(vii) Effects on cytokine-induced type II collagen degradation, MMP-mediated aggrecan degradation and aggrecanase-mediated degradation in bovine articular cartilage explants.

Results: Plant Extract Screening in BMP-2 Gene Reporter & Organ Culture

Tables 2 and 3 give details of the results of BMP-2 screening as follows:

15 extracts were found positive on BMP-2. From them, 5 confirmed hits and active subfractions were identified (Rosmarinus officinalis; Taraxacum officinalis, Lindera benzoin, Cyperus Rotundus, Iris pallida) and 5 further interesting positives (Rosmarinus officinalis, Carum carvi, Thymus vulgaris, Mentha spicata and Vitis vinifera) from 2 rounds of BMP-2 screening.

Confirmed hits and active subfractions (Rosmarinus officinalis, Taraxacum officinalis, Lindera benzoin, Cyperus rotundus, Iris pallida) and 5 further interesting positives (Rosmarinus officinalis, Carum carvi, Thymus vulgaris, Mentha spicata, Vitis vinifera) from 2 rounds of BMP-2 screening were also identified as active in the murine calvaria organ culture model.

Active extracts or subfractions were further confirmed to stimulate bone formation in the calvaria organ culture model: Iris pallida, Cyperus Rotundus, Rosmarinus officinalis (rosemary), Thymus vulgaris (thyme), Carum carvi (caraway).

TABLE 2
Summary of extracts positive in BMP-2 screen and
confirmed in organ culture

| | | | Active | |
|---|---|---|---|---|
| | | | concen- | Bone formation in |
| | | Ext | tration | organ culture: |
| Plant | Extract | n^{o} | (μg/ml) | Results & comments |
| Glycine max | EtOAc | 2001 | 10, 50 | Slight bone formation |
| (soy) | | | | |
| Rosmarinus | MeOH/water | 2004 | 10, 50 | Good bone formation in |
| officinalis | | | | calvaria culture |
| Rosmarinus | EtOAc | 2005 | 10 | Slight bone formation |
| officinalis | | | (50, toxic) | |
| Cyperus | Subfraction | 2012 | 10, 50 | Slight bone formation |
| rotundus | | | | |
| Iris pallida | C18 MeOH | 2022 | 10 | Some bone formation |
| | subfraction | | | subfraction |
| Thyme | EtOAc | 2067 | 10 | Some bone formation |
| Carvi | EtOAc | 2074 | 10 | Slight bone formation |

TABLE 3
Overall summary of plant extract results up to identification of hits

| Latin name | | | BMP-2 positive | BMP-2 positive | Organ |
|---|---|---|---|---|---|
| English name | Part | Extract | extracts | subfractions | culture |
| Glycine max | plant cell | ethylacetate | 768 | | 2001 |
| soybean | cultures | | | | |
| Acorus calamus | Tubers | MeOH/H₂O | 731 | | nd |
| Sweet flag | | | | | |
| Amelanchier | Fruit | MeOH/H₂O | 219 | | nd |
| ovalis | | | | | |
| Service berry | | | | | |
| Artemisia | Aerial | ethylacetate | 225 | | nd |
| vulgaris | parts | | | | |
| Mugwort/ | | | | | |
| wormwood | | | | | |
| Cyperus | Tubers | ethylacetate | 205, 2011 | 2012, 2013 | 2012 |
| rotundus | | | | | |
| nutgrass | | | | | |
| Taraxacum | Leaves | ethylacetate | 750, 2034 | 2035 | 2035 |
| officinalis | | | | | |
| dandelion | | | | | |
| Lindera benzoin | Aerial | ethylacetate | 740, 2059 | 2060 | 2059 |
| Spice bush | parts | | | | |
| Prunus persica | Seed | ethylacetate | 772 | | nd |
| peach | | | | | |
| Iris pallida | Tubers | MeOH/H₂O | 239, 760 | 760, 762, | 2022 |
| Sweet iris | | | | 2021, 2022 | |
| Rosmarinus | Leaves | MeOH/H₂O | 2004, 2127 | | 2004 |
| officinalis | | ethylacetate | 2005, 2089 | | 2005 |
| rosemary | | | | | |
| Carum carvi | Seeds | ethylacetate | 2074 | | 2074 |
| caraway | | | | | |
| Thymus vulgaris | Leaves | ethylacetate | 2067 | | 2067 |
| (thyme) | | | | | |
| Mentha spicata | Leaves | ethylacetate | 2072 | | 2072 |
| Spearmint | | | | | |
| Vitis vinifera | Fruit | ethylacetate | 2069 | | 2069 |
| grape | | | | | |
| Cichorium | leaves/ | MeOH/water | 2002 | | |
| intybus (chicory) | roots | EtOAc | 2003 | | |

| | |
|---|---|
| Anethum | not yet tested |
| graveolens (dill) | |
| Cuminum | |
| cyminum | |
| bixa orellana | |

Conclusions of Plant Extract Screening

BMP-2 hits confirmed in organ culture bone formation assay were extracts of soy seeds, rosemary leaves, thyme leaves, caraway seeds.

Example of Rosemary & Caraway Hits for BMP-2 Activity Confirmed in Alkaline Phosphatase Assay and Organ Culture

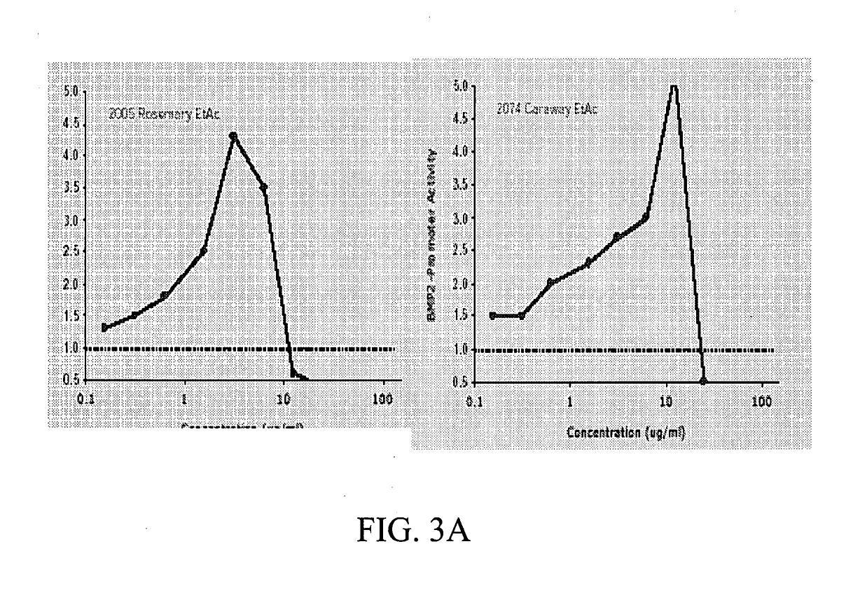

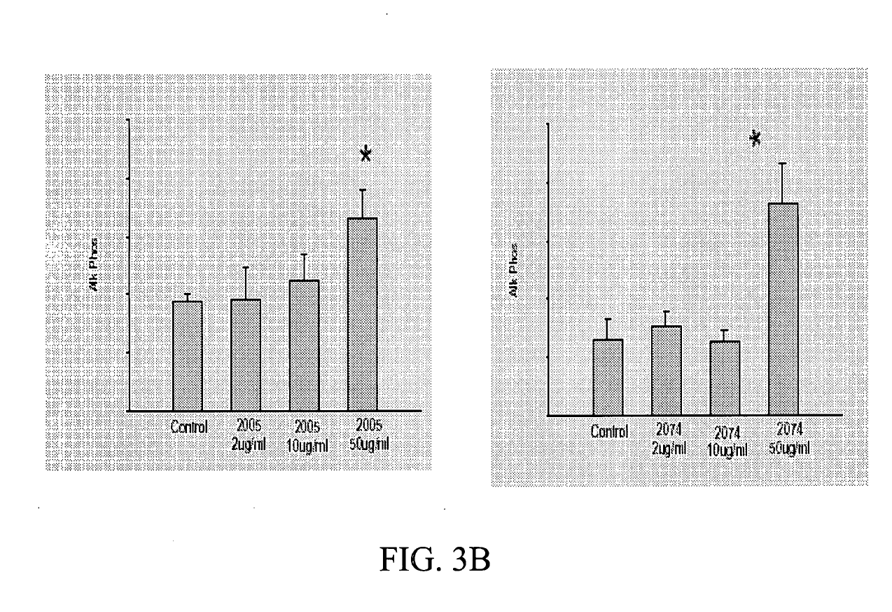

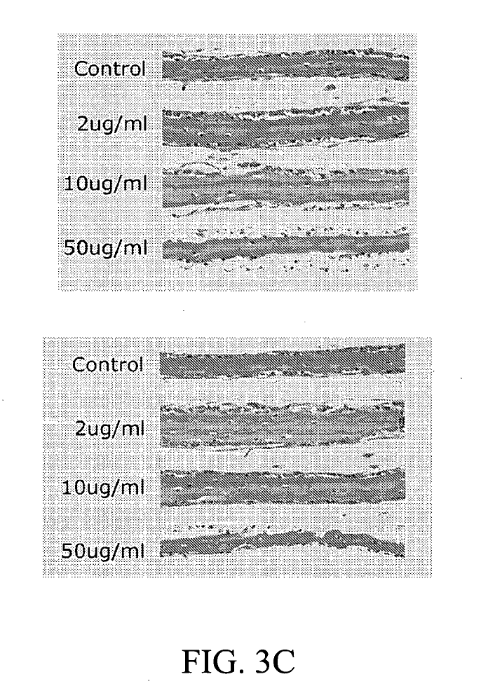

FIGS. 3A-C illustrate bone formation assay results for hits of rosemary and caraway extracts.

Effect of Extraction Procedure on BMP-2 Activity

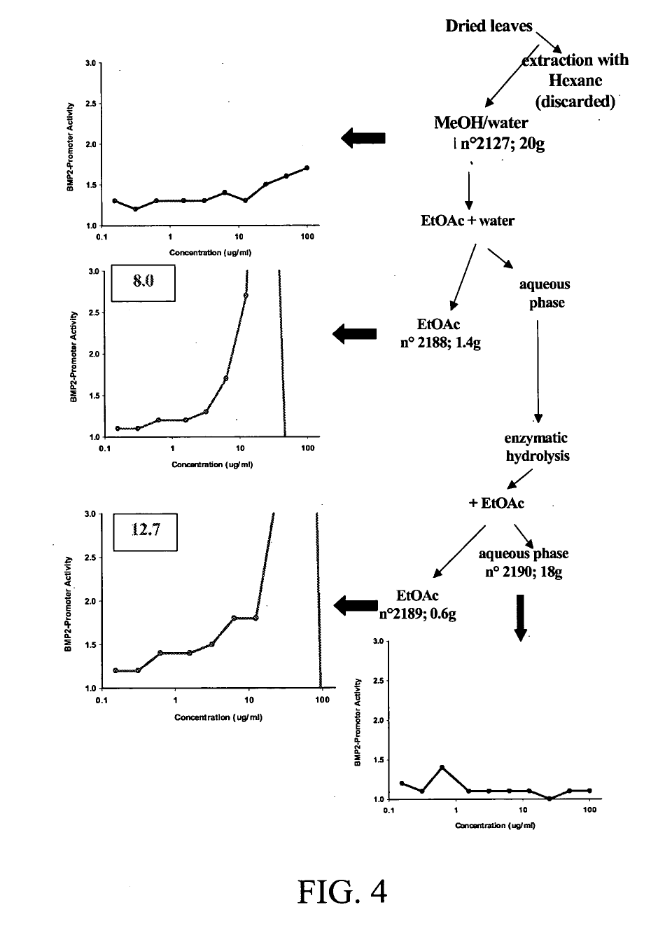

After a first extraction of rosemary with methanol/water on previously defatted leaves (ext. 2127), the induction of BMP-2 gene expression was 1.5× at 10 μg/ml (FIG. 4). A specific extraction of this extract, with ethyl acetate (2188), led to an increase in BMP-2 expression (8× induction). This suggests that the EtOAc extraction process resulted in the concentration of the active compounds from the original MeOH/water extract. After hydrolysis with glycosidases, the resulting ethyl acetate (2189) extract is also active, showing that additional active molecules have been extracted. Extract 2189 is even slightly more active than the non-hydrolyzed one.

These results show unambiguously an activity in both extracts (non hydrolyzed: 2188 and hydrolyzed: 2189) suggesting the existence of active molecules under two forms: free and/or bound (glycosylated) in the original extract.

Bone Formation in Calvaria Following Injection In Vivo

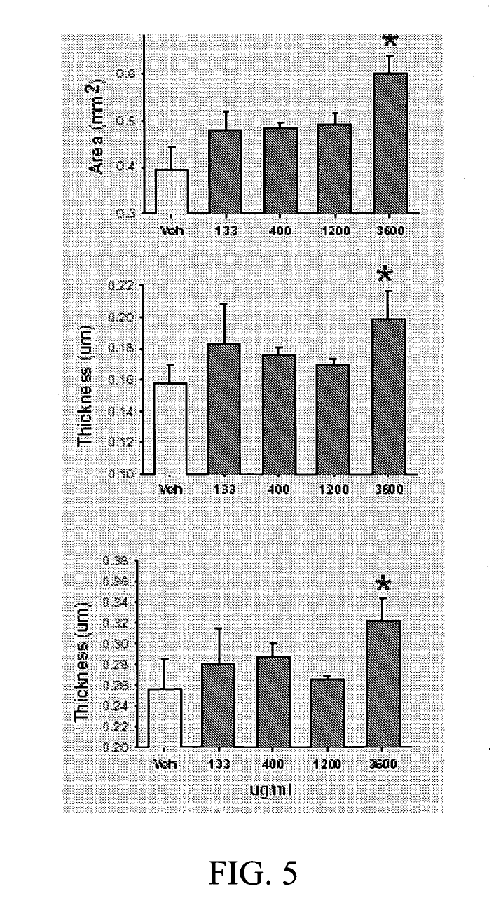

Rosmarinus officinalis (rosemary extract) shows bone formation activity in 3 independent bone formation in vitro assays (BMP-2, alkaline phosphatase, bone organ culture) as well as in the calvaria in vivo assay (see FIG. 5).

The rosemary extract (here the leaves were extracted first with water, and water extract was hydrolyzed then extracted with ethyl acetate) is injected into murine calvaria head, followed by ex vivo bone formation analysis.

Pure Compounds Screening

Phenolics were tested at concentrations of 1-10 μM

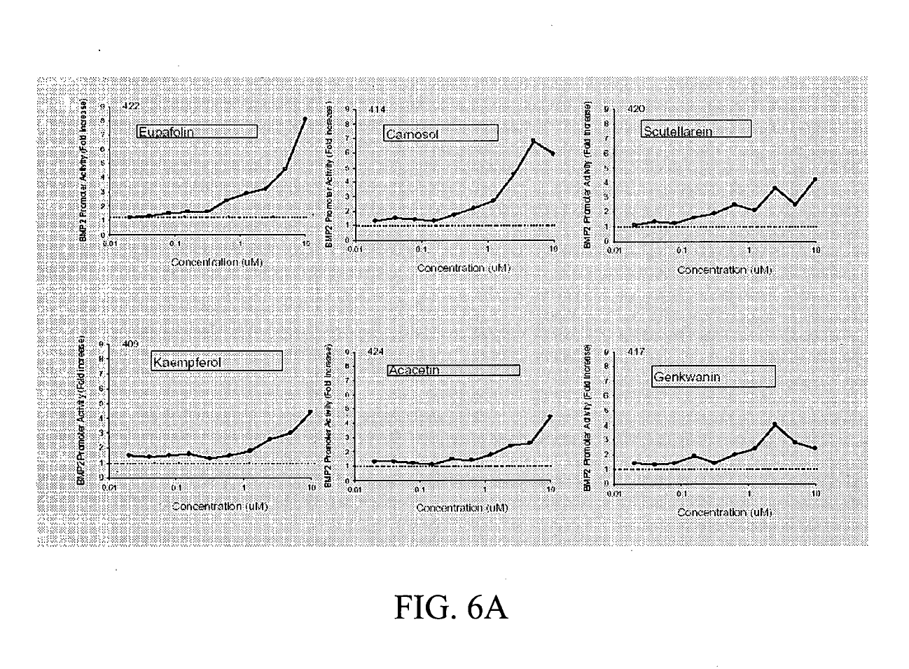

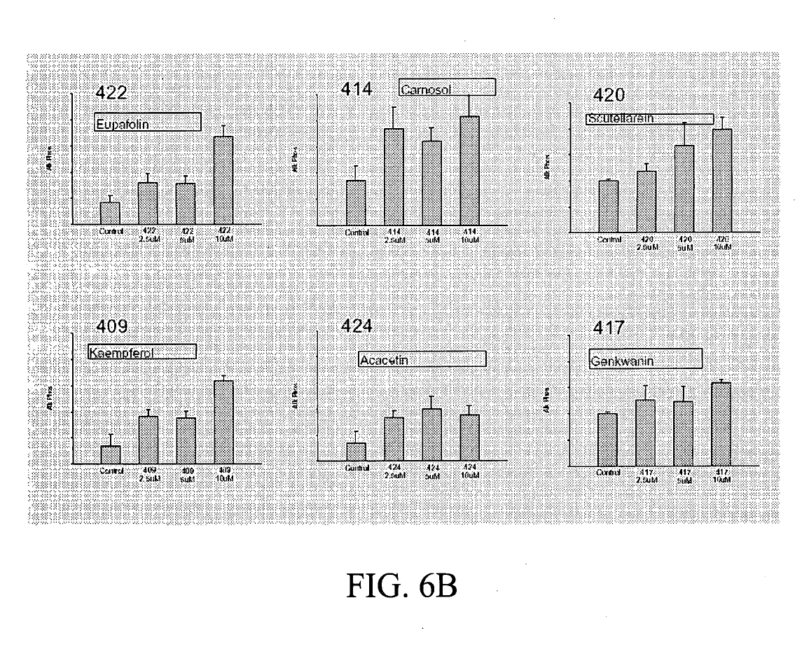

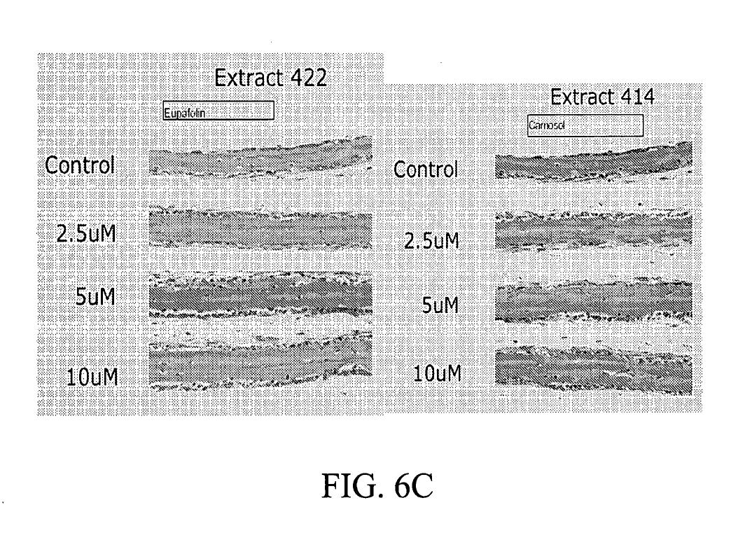

Phenolics active in BMP-2 assay are listed in Table 4. FIGS. 6A-C show certain phenolics positive in bone formation assays.

TABLE 4
Phenolics active in BMP-2, ALP and organ culture assays:

| | | ALP activity | Bone formation in |
|---|---|---|---|
| Flavonoid | BMP-2 induction | induction | organ culture |
| Eupafolin | 3-8X | 2X, 4X | Good formation for |
| | 2.5-10 μM | 5, 10 μM | 2.5, 5.0, 10 μM |
| Carnosol | 4.5-7X | 2X | Slight BF for 2.5 μM |
| | 2.5-10 μM | 2.5-10 μM | Good BF for 5, 10 μM |
| | | (not dose- | |
| | | responsive) | |
| Scutellarein | 3-4X | 2X | None at 2.5 μM |
| | 5, 10 μM | 10 μM | Some at 5, 10 μM |
| Genkwanin | 4X | 1.5X | none |
| | 5 μM | 10 μM | |
| Kaempferol | 2.5X | 2-4X | Slight at 10 μM |
| | 1-10 μM | 2.5-10 μM | |
| Acacetin | 3-5X | 2X | none |
| | 5-10 μM | 5-10 μM | |
| | | (not dose- | |
| | | responsive) | |

Rosemary Compositional Analysis

Table 5 shows pure compounds found in rosemary extracts.

TABLE 5
Rosemary Components

| Name | Class | Compound |
|---|---|---|
| Genkwanin | Flavone | Apigenin-7-methylated |
| Genkwanin-4′- | Flavone | Apigenin-7-methylated-4′- |
| glucoside | | glucoside |
| Salvigenin | Flavone | Polymethylated apigenin |
| Apigenin | Flavone | |
| Luteolin-3′-O- | Flavone | Luteolin-3′-O-glucuronide |
| glucuronide | | |
| Diosmetin | Flavone | Luteolin-3′-O-methylated |
| Luteolin | Flavone | |
| Hesperidin | Flavanone | Glycosylated hesperetin |
| Caffeic acid | Phenolic acid | |
| Carnosol | Phenolic diterpene | |
| Carnosic acid | Phenolic diterpene | |
| Rosmarinic acid | Phenolic acid | |

Analysis of BMP-2 Active Rosemary Extracts

The extract for analysis was chosen following previous results showing that the bone formation activity was concentrated in ethyl acetate extracts prepared from a methanol/water extract with (2189) or without enzymatic hydrolysis (2188), (see FIG. 4).

The ethyl extract acetate 2188 was selected for a phytochemical study of its main constituents including the identification and the purification of the compounds by HPLC/ELSD/UV/MS. This expertise was done by Analyticon Discovery GmbH, Postdam. An in-depth phytochemical evaluation was then completed on the Rosemary extract, active on BMP-2 assay. The preliminary results led to the isolation of 13 compounds. Nine compounds were identified. Four others required further investigations. Further studies describe the structural elucidation of these 4 compounds carried out through H-NMR and 2D-NMR (H, H-COSY, HMBC, HMQC) by Analyticon Discovery GmbH (Postdam).

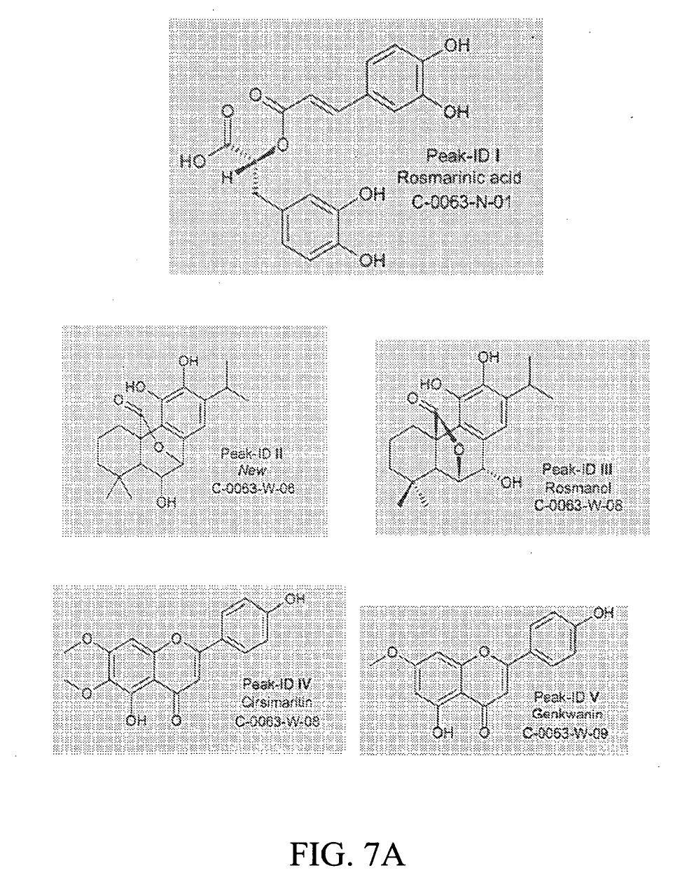

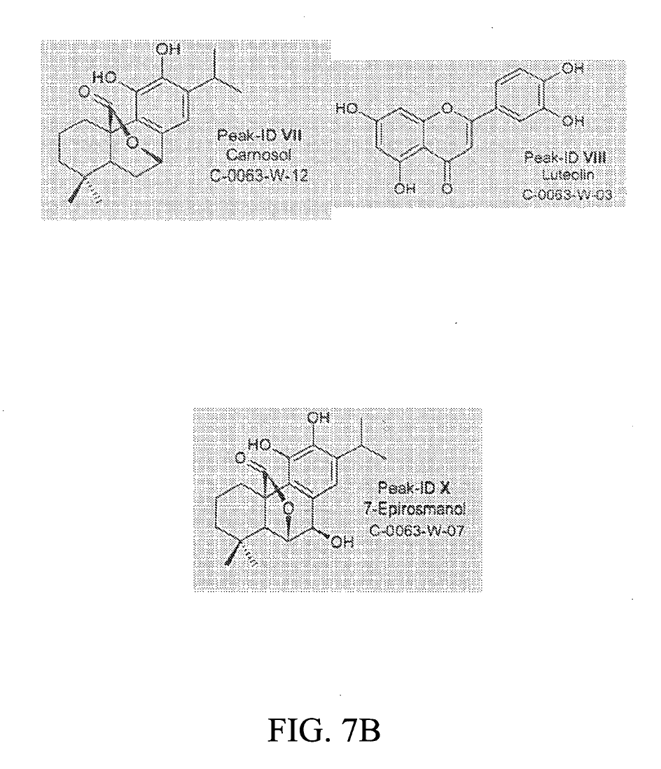

Analyticon provided thirteen pure identified molecules in order to carry out the evaluation of their biological activity on bone health. These compounds are listed in Table 6 and their structures illustrated in FIGS. 7A-B. Among them, three are new compounds, never described in literature (XI, XII and XIII). A correlation: chemical structure—biological activity of these 13 constituents, can provide an interesting and relevant tool for further development in bone health research.

TABLE 6
14 compounds isolated from rosemary extract 2188

| | | AMOUNT | | BMP- |
|---|---|---|---|---|
| N° | NAME | (mg) | PURITY (%) | Activity |
| I | Rosmarinic acid | 318 | 99.8 | NO |
| II | Isorosmanol | 73 | 99.7 | Not tested |
| III | Rosmanol | 14 | Mixture of III + | Not tested |
| | | | IV | |
| IV | Cirsimaritin | | | NO |
| V | Genkwanin | 7.9 | 94.1 | YES |
| VI | 7-Methyl rosmanol | 4.8 | 99.7 | Not tested |
| VII | Carnosol | 230 | 45 | YES |
| VIII | Luteolin | | | |
| IX | Scutellarein | not present in the extract | | YES |
| X | 7-Epirosmanol | 26 | 81 | Not tested |
| XI | Dehydroxy rosmarinic | 8.7 | 38 | Not tested |
| | acid | | | |
| XII | 6″-Feruloylnepitrin | 19.9 | 99.9 | Not tested |
| XIII | 6″-Coumaroylnepitrin | 13 | 94 | Not tested |
| XIV | Methylepirosmanol | 7 | 94 | Not tested |

Thirteen constituents of a Rosemary extract (2188) for which the structural elucidation has been achieved and validated are now available for the evaluation of their biological activity in bone health assays.

Rosemary Extract and its Anti Bone Resorptive Activity

Osteoporosis is a chronic disease characterized by a slow bone loss. Bone is not a dead tissue. On the contrary, it is constantly remodeled with old bone tissue being replaced by new one. This remodeling is controlled by osteoblasts, the cells that deposit bone and by osteoclasts, the cells that dissolves it. Usually, there is a tight coupling between bone formation and bone resorption so that no net bone loss occurs. In osteoporosis, this coupling is not perfect as bone loss is more prominent than bone formation. To treat osteoporosis, one can aim at increasing bone formation, at decreasing bone loss or both. In this example, it is shown that rosemary extracts can decrease bone loss.

Osteoclasts, differentiated from human Peripheral Blood Mononuclear Cells (PBMCs), were cultured on slices of bovine bones. Their resorbing activity was monitored by measuring the amount of type I collagen released in the media as they digest bone.

Type I collagen is the main organic molecule of bone. As bone is digested, the mineral phase of bone is dissolved exposing the collagen fibers to the proteolytic activity of matrix metalloproteinases. Once digested, the collagen fibers become soluble and are released in the culture media where their presence can be quantified by ELISA assays—CTX-I assay.

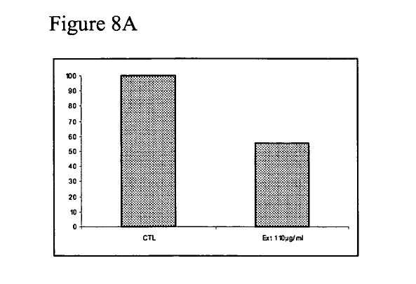

FIG. 8 A gives details of the effects of rosemary extract on the activity of human osteoclasts as follows: Rosemary extract 1 (extract P31 commersially available from Robertet) at a concentration of 10 μg/ml decreased the amount of type I collagen released from bone slices compared to culture media alone (control (CTL)) (FIG. 8A).

Rosemary Extract and Osteoarthritis

Osteoarthritis is a disease characterized by a slow destruction of articular cartilage. This cartilage destruction is due to an imbalance between the anabolic and catabolic activity of chondrocytes. The chondrocyte is the unique cell type present in cartilage and is responsible for the maintenance of the cartilage extracellular matrix. In osteoarthritis, catabolism is increased and is responsible for the cartilage loss. The extracellular matrix of cartilage is composed of 2 main molecules: type II collagen and aggrecan. While collagen is mainly digested by matrix metalloproteinases (MMPs), aggrecan can be degraded both by MMPs and another class of enzymes called aggrecanases.

We investigated here whether 2 different rosemary extracts and carnosol, one of the main components of this extract, could inhibit the degradation of collagen and aggrecan in cultured explants of articular cartilage. Explants from healthy bovine articular cartilage were put in culture. However, such explants naturally display a very low catabolic activity which is not ideal to test the anti-catabolic activity of potential bioactives. To improve the sensitivity of the assay, the explants are cultured in presence of 2 pro-inflammatory cytokines, TNF-α and oncostatin known to act together as potent inducers of MMP and aggrecanase activities.

Effects of Rosemary Extracts and Carnosol on Articular Cartilage Catabolism

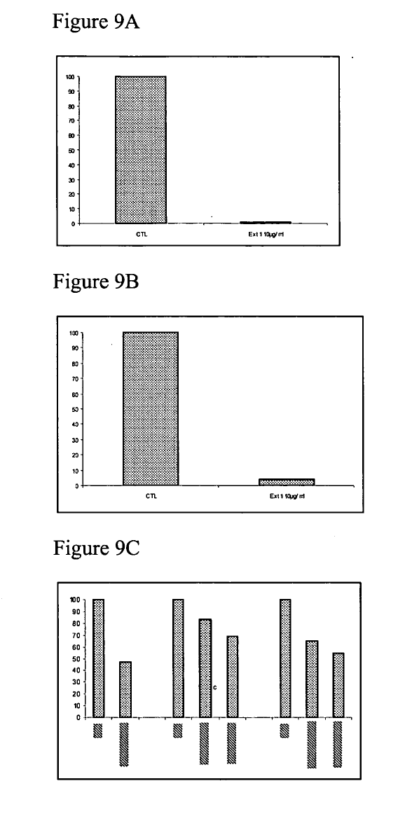

FIGS. 9 A, B and C give details on the effects of rosemary extract and carnosol on articular cartilage metabolism as follows: Rosemary extract 1 (extract P31 commercially available from Robertet) at a concentration of 10 μg/ml completely abrogated the collagen degradation induced by the pro-inflammatory cytokines TNF-α and oncostatin (control (CTL)) (FIG. 9A).

Rosemary extract 1 (extract P31 commercially available from Robertet) at a concentration of 10 μg/ml completely abrogated the MMP-mediated aggrecan degradation induced by the pro-inflammatory cytokines TNF-α and oncostatin (control (CTL)) (FIG. 9B).

Rosemary extract 1 (extract P31 commercially available from Robertet) at a concentration of 10 μg/ml also inhibited the aggrecanase-mediated aggrecan degradation induced by the pro-inflammatory cytokines TNF-α and oncostatin (control (CTL)). Similarly, another rosemary extract, rosemary extract 2 (from Nestec R&D Center in Tours obtained as described above) at a concentration of 1 or 5 μg/ml and carnosol at a concentration of 1 or 5 μM also inhibited cytokine induced collagen degradation (FIG. 9C).

OPN mRNA Induction:

Cell culture—HPOBTert osteoblasts were seeded on collagen-coated plates and grown in MEM Eagle α Modification medium supplemented with 10% fetal bovine serum, 1% L-glutamine and penicillin/streptomycin, 1 mM β-glycerolphosphate and 50 μg/ml ascorbic acid in a humidified atmosphere of 5% CO2, and 95% air at 37° C. When carnosol and inhibitors were added, an equivalent amount of Me₂SO was used as a vehicle control.

Analysis of mRNA levels by Real-Time PCR—Total cellular RNA was extracted using the NucleoSpin RNA II kit (Macherey-Nagel, Switzerland). Equal amounts (1 μg) of RNA from the different treatments were reverse-transcribed using the First Strand cDNA Synthesis kit for RT-PCR (Roche, Mannheim, Germany). For each sample, 2 μl 10× reaction buffer, 4 μl 25 mM MgCl2, 2 μl nucleotide mix, 2 μl random primers, 1 μl RNAse inhibitor and 0.4 μl AMV reverse transcriptase from the kit were added to the sample. The reverse transcriptase was performed at the following thermal cycling conditions (25° C. for 10 min, 42 C for 60 min, and 75° C. for 5 min) using the PTC-100TM Concept, Switzerland).

Real-time Quantitative PCR—Quantitative PCR was performed in 25 μl in triplicates. This consisted of 12.5 μl of Taqman 2× Universal PCR Master Mix, 1.25 μl Assay-on-Demand primers and probes (Applied Biosystems, USA) and 6.25 μl RNAse free water. Amplification was conducted in an ABI 7000 machine (Applied Biosystems) with the following thermal profile: 50° C. for 2 min, 10 min at 95° C., followed by 40 cycles of 95° C. for 15 s and 60° C. for 1 min. The gene expression levels were normalised to β-actin expression levels.

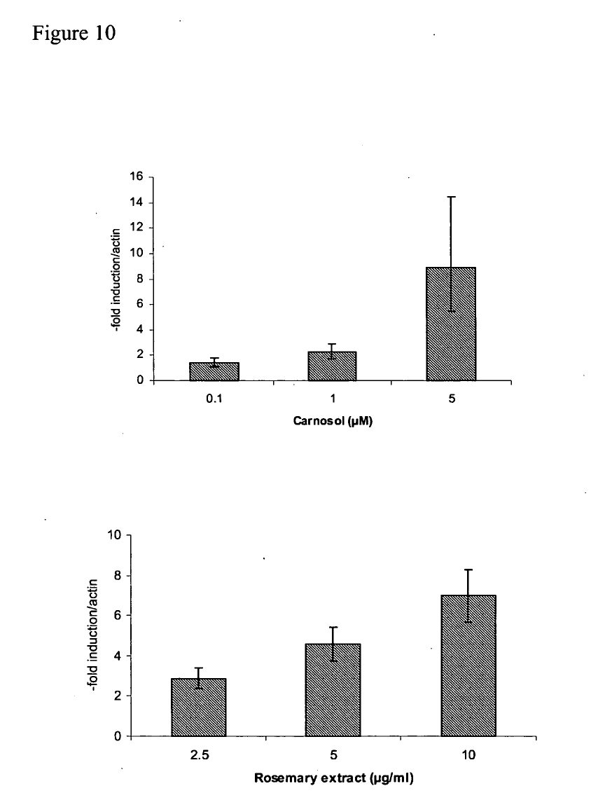

FIG. 10 shows that Rosemary extract or carnosol induce OPN expression dose-dependently by real-time PCR determination of OPN mRNA levels. HPOBtert cells were maintained for 48 h in rosemary extract or carnosol at the indicated doses.

NQO1 Induction

Preparation of cytoplasmic extracts—hPOBtert cells were washed two times with cold phosphate-buffered saline and harvested with lysis buffer (1% Triton X-100, 20 mM Tris/HCL pH 8, 137 mM Nacl, 10% Glycerol, 2 mM EDTA pH 8, and freshly added proteinase inhibitors: 1 mM phenylmethylsulfonylfluoride, 0.15 U/ml Aprotinin, 10 μg/ml Leupeptide and 10 μg/ml Pepstatin). The samples were centrifuged at 13000 rpm, 4° C. for 5 min and the supernatant transferred to a fresh tube. The protein concentration was determined using the BioRad protein assay. Approximately 50 μg of each sample were mixed with a suitable volume of sample buffer, denatured for 5 min at 95° C. together with 5 μl protein standard, chilled on ice, and loaded on a 10% ready gel, and submitted to immunoblot analysis using anti-NQO1 antibodies.

Immunoblotting—50 μg of protein cell lysate were resolved by SDS-PAGE. After electrophoresis, proteins were transferred to a PVDF membrane (Invitrogen) according to the manufacturer's protocol. Membranes probed for OPN and NQO1 were blocked and probed in 5% milk in Tris-buffered saline/Tween (20 mM Tris base, pH 7.6, 137 mM, 0.1% Tween 20). The blots were visualised by chemiluminescence development, Western blotting detection system (Amersham Biosciences).

Antibodies—The NQO1 (sc-16464)-specific antibodies were purchased from Santa Cruz Biotechnologies Inc (Santa Cruz, Calif.). The β-actin antibody (A-5441) was purchased from Sigma. The secondary antibodies were purchased from Sigma.

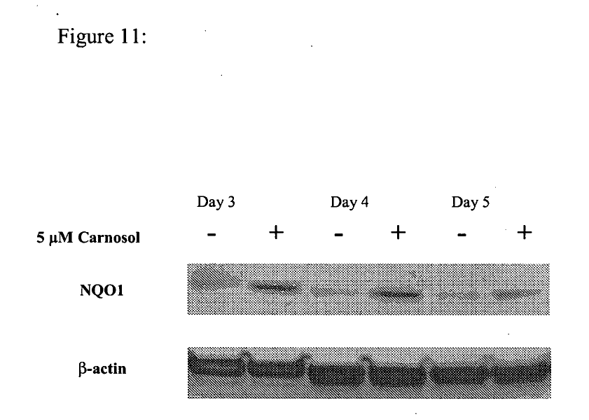

FIG. 11 shows that Carnosol induces the expression of the phase II enzyme NQO1, a typically Nrf-1 regulated gene/protein.

Safety Status

Tolerance testing

A tolerance test was performed in young male Sprague-Dawley rats. The rats were fed orally “by gavage” during 5 days with daily administration of 1 g (extract 2127, MeOH/water) per kg animal body weight. No abnormal behavior, mortality or signs of toxicitiy were observed during the treatment or the subsequent 10 days observation period. Rosmarinus officinalis was therefore considered as safe under these conditions.

### CONCLUSIONS

From 32 plant species, 120 extracts were screened in the BMP-2 assay, 15 were identified as positive hits. The most promising ones were from Rosmarinus officinalis (rosemary) together with those from Cyperus rotundus, Iris pallida, Thymus vulgaris (thyme) and Carum carvi (caraway), which were also found active in the murine calvaria organ culture model. Hits from the BMP-2 assay were confirmed in the alkaline phosphatase and organ culture functional assays for bone formation in vitro.

Rosmarinus officinalis (rosemary extract) was the most promising hit, showing bone formation activity in 3 independent bone formation in vitro assays (BMP-2, alkaline phosphatase, bone organ culture) as well as in the calvaria in vivo assay. For example, rosemary extracts stimulated bone formation following injection into murine calvaria in vivo.

Six phenolics that are components of rosemary (eupafolin, carnosol, scutellarein, genkwanin, kaempferol, acacetin) are active in the 3 bone formation assays. The most active ones are eupafolin and carnosol. Thirteen pure identified molecules were isolated from the active rosemary extract. Among them, 3 are new compounds (XI, XII and XIII), never described previously in the literature.

The presented data demonstrate furthermore that rosemary extracts and carnosol are able to slow down cartilage destruction. This property makes them interesting candidates to prevent osteoarthritis or slow down its progression in humans or pets.

The presented data also show that rosemary extract is able to increase bone formation but also to decrease bone resorption. It is not common to find a single compound/extract displaying both properties. This makes rosemary extract a highly interesting candidate to prevent osteoporosis or slow down its progression in humans or pets.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A composition comprising an active ingredient having a therapeutically effective amount of a plant or plant extract containing at least one phytochemical having the ability to induce bone morphogenic protein expression.

2. The composition of claim 1, wherein the plant or plant extract further inhibits bone resorption.

3. The composition of claim 1, wherein the plant is rosemary.

4. The composition of claim 1, wherein the phytochemical is selected from the group consisting of eupafolin, carnosol, scutellarein, genkwanin, kaempferol, acacetin and combinations thereof.

5. The composition of claim 1, which is in a form selected from the group consisting of a nutritionally balanced food, pet food, a dietary supplement, a treat, a pharmaceutical composition and combinations thereof.

6. The composition of claim 1, which is designed to achieve a result selected from the group consisting of assisting bone regeneration during fracture healing, increasing bone formation and bone mineral density during growth and optimizing peak bone mass or to decrease bone loss, in particular bone loss associated with age in humans or pets.

7. The composition of claim 1, which is designed to build cartilage and/or to prevent cartilage loss in humans or pets.

8. The composition of claim 1, which is designed to prevent osteoarthritis in humans or pets.

9. A composition comprising an active ingredient having a therapeutically effective amount of a rosemary plant or rosemary plant extract containing at least one phytochemical having the ability to induce bone morphogenic protein expression.

10. The composition of claim 9, wherein the phytochemical is selected from the group consisting of eupafolin, carnosol, scutellarein, genkwanin, kaempferol, acacetin and combinations thereof.

11. A method for manufacturing a food composition comprising
providing a food composition; and
adding to the food composition an active ingredient having a plant or a plant extract containing at least one phytochemical having the ability to stimulate bone morphogenic protein and/or inhibit bone resorption to prepare the composition.

12. A method according to claim 11, wherein the plant is rosemary.

13. A method according to claim 11, wherein the phytochemical is selected from the group consisting of eupafolin, carnosol, scutellarein, genkwanin, kaempferol, acacetin and combinations thereof.

14. The method of claim 11, wherein the composition includes components selected from the group consisting of chicory, tea, cocoa, bioactives, antioxidants, fatty acids, prebiotic fibers, glucosamine, chondroitin sulphate and combinations thereof.

15. A method for the treatment, alleviation or prevention of bone disorder or maintenance of bone health, the method comprising
administering a therapeutically-effective amount of a composition comprising an active ingredient having an effective amount of at least one plant or plant extract containing at least one phytochemical having the ability to induce bone morphogenic protein expression to an individual in need of same.

16. A method according to claim 15, wherein the plant or plant extract further inhibits bone resorption.

17. A method according to claim 15, wherein the plant is rosemary.

18. A method according to claim 15, wherein the phytochemical is selected from the group consisting of eupafolin, carnosol, scutellarein, genkwanin, kaempferol, acacetin and combinations thereof.

19. A method of increasing bone formation, bone mineral density during growth and optimize peak bone mass in humans or pets, the method comprising
feeding an individual, a composition comprising an active ingredient having a therapeutically effective amount of at least one plant or plant extract containing at least one phytochemical having the ability to induce bone morphogenic protein expression.

20. A method according to claim 19, wherein the plant or plant extract further inhibits bone resorption.

21. A method according to claim 19, wherein the plant is rosemary.

22. A method according to claim 19, wherein the phytochemical is selected from the group consisting of eupafolin, carnosol, scutellarein, genkwanin, kaempferol, acacetin and combinations thereof.

23. A method for the treatment, alleviation and/or prophylaxis of osteoarthritis in pets and humans, the method comprising
feeding an individual having or at risk of osteoarthritis, a composition comprising an active ingredient having a therapeutically effective amount of at least one plant or plant extract containing at least one phytochemical having the ability to induce bone morphogenic protein expression in the individual.

24. A method according to claim 23, wherein the plant or plant extract further inhibits bone resorption.

25. A method according to claim 23, wherein the plant is rosemary.

26. A method according to claim 23, wherein the phytochemical is selected from the group consisting of eupafolin, carnosol, scutellarein, genkwanin, kaempferol, acacetin and combinations thereof.

27. A method of treating or preventing osteoporosis, the method comprising
administering to an individual having or at risk of osteoporosis a therapeutically effective amount of a composition comprising an active ingredient having an effective amount of at least one plant or plant extract containing at least one phytochemical having the ability to induce bone morphogenic protein expression in the individual.

28. A method of stimulating bone regeneration during fracture healing, the method comprising
feeding an individual having a fracture, a therapeutically-effective amount of a composition comprising an active ingredient having an effective amount of at least one plant or plant extract containing at least one phytochemical having the ability to induce bone morphogenic protein expression in the individual.

29. A method of decreasing bone loss, the method comprising
feeding an individual exhibiting a bone loss, a composition comprising an active ingredient having a therapeutically effective amount of at least one plant or plant extract containing at least one phytochemical having the ability to induce bone morphogenic protein expression in the individual.
